(19) United States
(12) Patent Application Publication
(10) Pub. No.: US 2018/0059100 A1
(43) Pub. Date: Mar. 1, 2018

(54) CARBON NANOTUBE-BASED MAGNETIC BIO-INK AND BIOSENSORS AND METHODS OF MAKING AND USING

(71) Applicant: McMaster University, Hamilton (CA)

(72) Inventors: Abdel Rahman Abdel Fattah, Etobicoke (CA); Ahmed M. Abdalla, Cairo (EG); Fei Geng, Hamilton (CA); Sarah Mishriki, Scarborough (CA); Elvira Meleca, Hamilton (CA); Ishwar K. Puri, Ancaster (CA); Suvojit Ghosh, Hamilton (CA)

(21) Appl. No.: 15/692,006

(22) Filed: Aug. 31, 2017

Related U.S. Application Data
(60) Provisional application No. 62/381,611, filed on Aug. 31, 2016.

(51) Int. Cl. G01N 33/543

(52) U.S. Cl. CPC G01N 33/5438; G01N 2333/4703; G01N 33/54333; G01N 33/54346

(57) ABSTRACT

A new magnetic carbon nanotube (mCNT) biosensor ink (mbio-ink) that utilizes immobilized capture agents to detect specific target analytes via a simple current response method is presented here, expanding the applications of magnetic carbon nanotubes as biomolecule-sensing nanomaterials. The employment of a magnetic field to print the mbio-ink into electrically conductive networks facilitates its integration with an external circuit via the use of a simple electrode system. When the sensor is connected to an external power source, the reduction in current, caused by an interaction between the capture agents and target analyte, is detected. The rapid detection technique and generic benchtop fabrication method allows for scale up, while the small volumes required and magnet independent electrical measurements renders the mbio-ink attractive for drug screening and disease detection applications.

The present application claims the benefit of provisional patent application No. 62/381,611, filed Aug. 31, 2016, the contents of which are herein incorporated by reference.

### FIELD

The present application relates to the field of biological sensing. More particularly, the present invention is in the technical field of biological sensor design and fabrication.

### BACKGROUND

Early pathogen detection and diagnosis of disease contributes to the containment and prevention of disease spread. Regularly used strategies of antigen (Ag) detection include long-standing laboratory methods such as enzyme-linked immunosorbent assay (ELISA) and polymerase chain reaction (PCR). Both of these methods have limitations that include being time consuming and labor intensive.

For these reasons, advances in biosensing nanomaterials and nanofabrication have been leveraged to efficiently, inexpensively and rapidly identify pathogen and disease biomarkers, thus replacing traditional detection methods. Carbon nanotubes (CNTs), which include single-walled, double-walled and multi-walled carbon nanotubes, have been identified as an ideal material for use in modern biosensing applications, as they typically exhibit favorable electrical and electrochemical properties that make them useful biological and chemical sensors. In addition, the ability of CNTs to interact with a variety of different chemical moieties allows one to control the selectivity of a CNT-based biosensor. Functionalization of CNTs, such as multi-walled carbon nanotubes (MWCNTs) facilitates the covalent attachment of antibodies and other recognition molecules that selectively interact with the target agents in solution. This interaction has been measured previously using various electrochemical methods such as cyclic voltammetry or amperometry to detect the presence of the target molecule in a sample. Such tests depend on reversible surface reactions which take place when current is supplied to the carbon nanotube and is dependent upon the current direction. The response is therefore removed from directly monitoring the binding kinetics of the Ags and Abs. Current changes under these cyclic conditions correspond to unique surface reactions allowing for the identification of the targets and their concentrations. However, additional reagents and preparation steps have been used in order to allow for the surface reactions to take place and sophisticated electrical equipment has been required to supply and analyze biosensor responses.

Magnetic nanoparticles (MNPs) have been patterned into a polymer matrix to produce a functional material.¹⁻¹² MNPs have also been used to guide the deposition of CNTs by remotely manipulating MNP-CNT complexes with a magnetic field, for instance, to print conductive networks and sensors.^{1, 13} Magnetic CNTs (mCNTs or CNT-Fe₃O₄ hybrid nanoparticles)) have been explored for biological applications, e.g., human IgG immunosensors,² and for electrical current measurements.^{14, 15} Most such measurements involve cyclic voltammogram analysis, which requires sophisticated acquisition devices and a magnet to be continually present during sensing to affix the sensing material to an electrode.¹⁶

### SUMMARY

A low cost method of sensing antigens and other target biomolecular markers in real time is disclosed herein. The method utilizes an easily fabricated electrode that can rapidly detect the presence of target analytes, such as biomolecules (e.g. antigens) and cells, in solution. In an exemplary embodiment, a new magnetic multi-walled carbon nanotube (mMWCNT) biosensor ink (mbio-ink) that comprises immobilized antibodies to detect specific antigens (Ags) within 60 seconds by measurement of a simple current decrease across an electrode is described in the present application. This biosensor demonstrated a novel transient current response to the binding of Ags for a given Ab, making the detection of Ags simple, effective, and immediate relative to the existing electrochemical methods discussed above. Unlike conventional measuring techniques, this new biosensor composed of the mbio-ink conveys real-time Ag-Ab binding kinetics and directly offers a corresponding electrical response. The transient monitoring of current changes provides a means to semi-quantitatively evaluate the concentration of the target within sixty seconds without the need of any additional reagents or complicated electrochemical testing equipment. Furthermore, the ease of fabrication of the biosensor ink is facilitated by a novel magnetic printing method described herein, thus providing a simple, low-cost manufacturing process. The ink is printed using an external magnet by dynamically self-organizing its nanoparticle constituent into an electrically conducting strip in 4-5 minutes, excluding drying time. The resulting biosensor detects Ag samples with picomolar sensitivity in less than a minute. The ease of fabrication, detection and low cost of the invention described herein make it an ideal tool for the detection of antigens and other biomolecules in real time.

Accordingly, in one aspect the present application includes a biosensor for detecting a target analyte in a sample comprising:
an external circuit;
a sensing electrode that is electrically connected to the external circuit;
the sensing electrode comprising carbon nanotubes (CNTs) functionalized with magnetic nanoparticles (MNPs) and one or more capture agents; and
a detector that detects a change in current through the sensing electrode resulting from a selective binding interaction between the one or more capture agents and the target analyte.

In a further aspect of the present application, there is included a method of making a biosensor comprising:
(1) preparing a magnetic bio-ink an aqueous solution of mCNTs functionalized with one or more capture agents by:
   (a) treating CNTs with an oxidizing agent to form reactive functional groups selected from carboxylic acids, aldehydes and alcohols on a surface of the CNTs to provide activated CNTs (aCNTs);
   (b) combining the aCNTs with magnetic nanoparticles to provide mCNTs comprising unreacted carboxylic acids, aldehydes and alcohols;
   (c) combining the mCNTs with a capture agent comprising one or more functional groups that form a covalent bond with the unreacted carboxylic acids, aldehydes and alcohols; and
   (d) treating the mCNTs from (c) with a blocking agent;
(2) depositing the magnetic bio-ink onto a substrate;
(3) forming the magnetic bio-ink into a sensing electrode located in a position electrically connected to an external circuit using an external magnet; and
(4) allowing the magnetic bio-ink to dry and removing the external magnet.

In another aspect, the present application includes a method of detecting a target analyte comprising:
(a) depositing a sample suspected of comprising the target analyte onto the sensing electrode of a biosensor of the application; and
(b) observing the current through the sensing electrode using the detector,
   wherein a change in current through the sensing electrode in the presence of the sample compared to a control indicates that the same contains the target analyte.

The present application also includes an electrode composed exclusively of multiwall carbon nanotubes (MWCNTs) functionalized with COOH, C═O, and C—OH group; and a magnetic nanoparticle and a protein, such as an antibody, covalently bonded to the MWCNT via COOH, C═O, and C—OH functional groups;

a detector device capable of rapidly (eg. <60 s) detecting the presence of a target analyte (eg. antigen, protein, DNA, molecule, etc.), without the use of a redox mediator or reporter molecule, and relating it to a change in current through the electrode resulting from the selective binding interaction between the covalently bonded protein (eg. antibody) and the target analyte (eg. complementary antigen). In some embodiments, the electrode is printed onto the detector circuit by depositing the MWCNTs with antibodies and magnetic nanoparticles directly attached to their surface in solution onto a substrate with the precise location of the electrode defined by a magnetic field produced temporarily by a magnet underneath this location.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF FIGURES

The embodiments of the application will now be described in greater detail with reference to the attached drawings in which:

FIG. 1 shows an exemplary c-Myc biosensor 100 of the application. Multiwalled CNTs 140 are treated with concentrated nitric acid, which produces carboxylic acid (—COOH), and other, active groups on the outer surfaces of the MWCNT. Some of these active groups act as nucleation sites for in situ co-precipitation of magnetite (Fe₃O₄) nanocrystals 150 while the remainder remain available for forming covalent bonds with anti-c-Myc 160 amine (—NH₂) groups. The MWCNT surfaces are blocked using a blocking agent 170 (e.g. Tween™ 20) to prevent non-specific interactions of Ag with the MWCNT surface. The magnetic biological ink 130 is then deposited on a glass substrate 120 using a pipette where it self organizes under the influence of an external magnetic field 180 (not shown) that guides the ink to print a dense electrically conducting strip 110. After the ink dispersant evaporates, the sensor strip is connected to an external circuit using electrodes 200. When an aqueous c-Myc solution 250 is deposited on this strip, its electrical resistance increases. A programmable logic controller measures the current 190 flowing through this patterned strip.

FIG. 2 shows the X-Ray Diffraction Analysis of exemplary mMWCNTs. The XRD (Co K_{α}, λ=1.79 Å) patterns for magnetite to MWCNTs weight ratios γ=0.1, 0.2, and 0.4 confirm the presence of a magnetite (Fe₃O₄) phase and a hexagonal carbon phase that originates from the carbon nanotubes.

FIG. 3 shows Transmission Electron Microscopy (TEM) TEM images of exemplary mMWCNT samples at various magnetization weight ratios γ=0.1 to 0.4 (from top to bottom) confirm that all samples have been successfully decorated with highly crystalline MNPs that are synthesized within a narrow size distribution around ˜10 nm.

FIG. 4 shows the magnetic behavior of exemplary mbio-inks with varying Fe₃O₄ to MWCNT weight ratios. Magnetic hysteresis curves show that all magnetized MWCNT samples exhibit superparamagnetic behaviour, but have different saturation values Mₛ depending on γ. The greater the magnetite content, the stronger is the material response to a magnetic field.

FIG. 5 shows the visualization of Ab immobilization on the surface of exemplary magnetic MWCNTs. First, FITC-labeled fluorescent Abs were employed to confirm Ab immobilization on the surface of magnetic MWCNTs for an Ab:MWCNT weight ratio β=2.5×10⁻⁴ in an ink where Abs are covalently bonded with magnetized MWCNTs that have Fe₃O₄:MWCNT weight ratios (a) γ=0.1, (b) γ=0.2 and (c) γ=0.4, and (d) for an ink that contains Abs and MNPs, which were adsorbed on the surface of MWCNTs with γ=0.4. For (e) β=0, γ=0.4, and no fluorescence is observed from MWCNTs and Fe₃O₄, confirming that the fluorescence observed in (a)-(d) originates from FITC-labeled Abs only. No visual differences in fluorescence was detected for samples containing different weight ratios of magnetite, and those containing adsorbed and covalently bonded immobilized Abs and MNPs. For (a)-(e), the images on the left column are optical bright field and the images on the right column are bright field and fluorescent overlay. (f) STEM and EELS micrographs reveal the presence of elemental carbon (C), oxygen (O) and nitrogen (N). The nitrogen, which is present only in Anti-c-Myc Abs, confirms Ab immobilization on the conductive MWCNT network.

FIG. 6 (a) shows an exemplary printing technique and assembly of an exemplary biosensor. 10 μL of the magnetic bio-ink 130 is deposited on top of a glass coverslip 120 that is placed on a permanent magnet 180. The applied magnetic field concentrates and self organizes the functionalized magnetic MWCNTs on the substrate. After the supernatant in the ink is evaporated, a patterned strip of densely packed Ab-functionalized MWCNTs remains deposited on the substrate, which forms the sensing electrode 110. The STEM micrograph identifies MNPs and anti-c-Myc that constitute the print based on the magnetic bio-ink. Electrodes 200 are readily connected to either end of the strip, providing current 190 to the sensor; (b) shows an exemplary voltage divider 230 with a reference resistor R_{ref}=100 kΩ which monitors current changes that measure the biosensor responses to the various samples that are deposited on it.

FIG. 7 (shows the transient current response of an exemplary biosensor upon addition of c-Myc in varying concentrations, BSA and deionized (DI) water. Immediately after 2 μL samples are deposited on the sensor strip, the DI water and BSA samples induce a rapid decrease in electrical current, which subsequently levels out. In contrast, since the sensor is inherently sensitive to c-Myc Ag interactions due to the anti-c-Myc Abs that are covalently bonded to the surfaces of MWCNTs, the current for all c-Myc samples decreases to levels below those measured for DI water and BSA deposition, which offers proof of targeted detection and sensor specificity to c-Myc Ags. The higher the c-Myc concentration in a sample, the larger the current decrease it induces. All of the deposited c-Myc samples produce a steady current decrease during the period 30 s<t<60 s. Normalizing the average current over that duration leads to the quasi-linear response shown in (c); and (d) shows that there is a linear correlation between the normalized current gradients in (c) and the corresponding c-Myc concentrations.

FIG. 8 shows that after the deposition of 5 successive 1 μL samples of BSA [40 pM], an exemplary biosensor response is relatively unchanged. Only after the introduction of c-Myc [40 pM] samples is the reduction in current observed, validating the specificity of the exemplary sensor to the target antigen in the presence of another protein.

FIG. 9 shows the response of an exemplary biosensor to successive 1 μL sample addition of various solutions to (i) the functionalized mMWCNT without antibodies, (ii) MWCNTs with antibodies and magnetic nanoparticles adsorbed on the surface, and (iii) the mbio-ink. (a) Acting as a control, magnetized MWCNTs that are not functionalized with Abs (i) cannot distinguish between 40 pM BSA and c-Myc samples, black dashed and solid curves respectively. When anti-c-Myc is immobilized on the MWCNT surfaces through adsorption, again (ii) there is insufficient discrimination between these two samples. In contrast, a sensor fabricated using an ink in which anti-cMyc is attached to the MWCNT surfaces through acid functionalization, (iii) clearly distinguishes between the two negative control samples, DI water and BSA, and the Ag of interest, c-Myc.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “an antibody” or “magnetic nanoparticle” should be understood to present certain aspects with one substance or two or more additional substances.

In embodiments comprising an “additional” or “second” component, such as an additional or second antibody, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “sample(s)” as used herein refers to any material that one wishes to assay using the biosensor of the application. The sample may be from any source, for example, any biological (for example human or animal medical samples), environmental (for example water or soil) or natural (for example plants) source, or from any manufactured or synthetic source (for example food or drinks). The sample is one that comprises or is suspected of comprising one or more target analytes.

The term “nucleic acid” refers to polynucleotides such as deoxyribonucleic acid (DNA) and ribonucleic acid (RNA).

The term “aptamer” as used herein refers to short, chemically synthesized, single stranded (ss) RNA or DNA oligonucleotides which fold into specific three-dimensional (3D) structures that bind to a specific target analytes with dissociation constants, for example, in the pico- to nano-molar range.

The term “printing” as used herein refers to the placement of a substance, such as a magnetic bio-ink of the application, on a substrate using a mechanical device that prints the substance onto the substrate.

The term “blocking agent” as used herein refers to an agent that is added to the sensing electrode comprising carbon nanotubes (CNTs) functionalized with magnetic nanoparticles (MNPs) and one or more capture agents, to prevent non-specific interactions of analytes, other than the target analyte, with the surface of the CNTs.

The term “carbon nanotubes” or “CNT” includes single-walled, double-walled and multi-walled carbon nanotubes.

### II. Description of Various Embodiments of the Application

In an exemplary embodiment of the present application, a magnetic field was employed to print a magnetic ink comprising capture agents immobilized on multi-walled carbon nanotubes (MWCNTs) as an electrically conducting sensing electrode, which is integrated into an external circuit, for example, using simple electrodes. Placing a sample containing target analytes on the sensing electrode initiates specific interactions between the target and the capture agent, reducing the electric current passing through the sensing electrode. The current reduction, which is the target detection signal, is measured using, in this exemplary embodiment, a programmable logic controller (PLC).

Fabrication of the biosensors of the application is inexpensive, signal detection is rapid, and the benchtop fabrication method is scalable since the ink can be readily integrated into standard inkjet and 3D printers. The small ink volumes required for biosensor fabrication and the magnet-independent electrical measurements make the ink attractive for integration into, for example, drug screening and disease detection applications.

Accordingly, the present application includes a biosensor for detecting a target analyte in a sample comprising:
an external circuit;
a sensing electrode that is electrically connected to the external circuit;
the sensing electrode comprising carbon nanotubes (CNTs) functionalized with magnetic nanoparticles (MNPs) and one or more capture agents; and
a detector that detects a change in current through the sensing electrode resulting from a selective binding interaction between the one or more capture agents and the target analyte.

In some embodiments, the biosensor further comprises a transmitter for sending data obtained by the biosensor to a remote sensor and a power source.

In some embodiments, the external circuit is a voltage divider circuit.

In some embodiments the CNTs have been activated, that is, treated under conditions, to provide functional groups on at least part of the surface of the CNTs for attachment of the magnetic nanoparticles and capture agents. In some embodiments, the functional groups are selected from one or more of carboxylic acids (—COOH), aldehydes (—C(O)H) and alcohols (—OH). In some embodiments, the conditions comprise treating the MWCNTs with an oxidizing agent. In some embodiments, the conditions comprise treating the CNTs with a strong acid, such as concentrated nitric acid (HNO₃).

In some embodiments the magnetic nanoparticles are any magnetic nanoparticles that are not toxic or that do not degrade any biological material in the sensor and that have sufficient magnetic properties to allow for printing and localization on a substrate using a magnet. The magnetic nanoparticles should also react with the activated CNTs in a manner that leaves a sufficient amount of active sites open for capture agent binding. In some embodiments, the magnetic nanoparticles are magnetite nanocrystals (Fe₃O₄).

In some embodiments, the CNTs are multiwalled carbon nanotubes (MWCNTs).

In some embodiments, the Fe₃O₄:CNT weight ratio (γ) is from about 0.05 to about 1. In some embodiments, γ is about 0.1 to about 0.5. In some embodiments γ is about 0.4.

In some embodiments, the capture agent:CNT weight ratio (β) is about 2.5×10⁻² to about 2.5×10⁻⁴, or about 2.5×10⁻⁴.

In some embodiments, the target analyte is selected from a biomolecule and any material comprising a biomolecule, such as tissues, cells, cell lysates, bodily specimens and environmental specimens. In some embodiments the biomolecule is selected from proteins, peptides and nucleic acids (DNA or RNA). In some embodiments, the target biomolecule is selected from an antibody, antigen, enzyme, aptamer and receptor.

In some embodiments, the capture agent is any molecule that contains a functional group that will covalently bond to the activated CNTs and that will specifically interact with the target analyte so that the target analyte becomes immobilized on the sensing electrode. Accordingly, the capture agent will depend on the identity of the target analyte. For example, if the target analyte is an antigen, the capture agent will be an antibody that specifically binds to that antigen.

In some embodiments, the capture agent is an aptamer, such as a DNA or RNA aptamer, that has been prepared to specifically recognize and bind to the target analyte.

In some embodiments, the target analyte is an antibody and the capture agent is the antigen that specifically binds to that antibody.

In some embodiments, the target analyte is an antigen and the capture agent is the antibody that specifically binds to that antigen. In some embodiments, the target analyte is an antigen that is associated with, or diagnostic of, a particular disease, such as cancer.

In some embodiments, the capture agent is a receptor and the target analyte is a ligand that specifically binds to that receptor.

In some embodiments, the sensing electrode consists essentially of multiwall carbon nanotubes (CNTs) functionalized with magnetic nanoparticles (MNPs) and one or more capture agents. In some embodiments the multiwall carbon nanotubes (CNTs) functionalized with magnetic nanoparticles (MNPs) and one or more capture agents are treated with a blocking agent. In some embodiments the blocking agent is a surfactant. In some embodiments the blocking agent is a polysorbate (e.g. Tween™20). In some embodiments the blocking agent is bovine serum albumin (BSA)

In some embodiments, the detector is a programmable logic controller (PLC).

In some embodiments, the sensing electrode is located between, and is in contact with, two further electrodes. In some embodiments, the sensing electrode is located so that it bridges a gap between the two further electrodes to complete the external circuit.

In some embodiments, the biosensor further comprises a support and the sensing electrodes, and any further electrodes are on a surface of the support. In some embodiments, the support is made of any non-conducting material. In some embodiments the support is made of glass or any plastic material.

In some embodiments, the sensing electrode is printed onto the support by depositing a solution of the magnetic CNTs onto the substrate and a magnate located on the opposite side of the substrate is used to position the CNTs, wherein the magnet is removed following positioning. In some embodiments, the sensing electrode is positioned into a dense electrically conducting strip.

In some embodiments an adhesive substance is utilized to immobilize the CNTs to the surface of the substrate. Any suitable adhesive can be used provided it does not interfere with the electrical conductivity of the biosensor and it does not interact adversely with any of the components of the biosensor.

In all embodiments, a magnet is not used in the biosensor of the application during detection of the target analyte.

In some embodiments, the further electrodes are comprised of a conductive layer that is printed onto are least a portion of a surface of the support. In some embodiments, the conductive layer is comprised of a polymeric organosilicon compound, such as polydimethylsiloxane. In some embodiments, the insulating layer forms a pair of electrodes with an insulating gap that is bridged by the sensing electrode. In some embodiments, the conductive layer further comprises a conducting metal, such as aluminum foil.

In some embodiments, the change in current through the sensing electrode resulting from a selective binding interaction between the one or more capture agents and the target analyte is a decrease in current. In some embodiments, the decrease in current is proportional to the concentration of the target analyte in the sample.

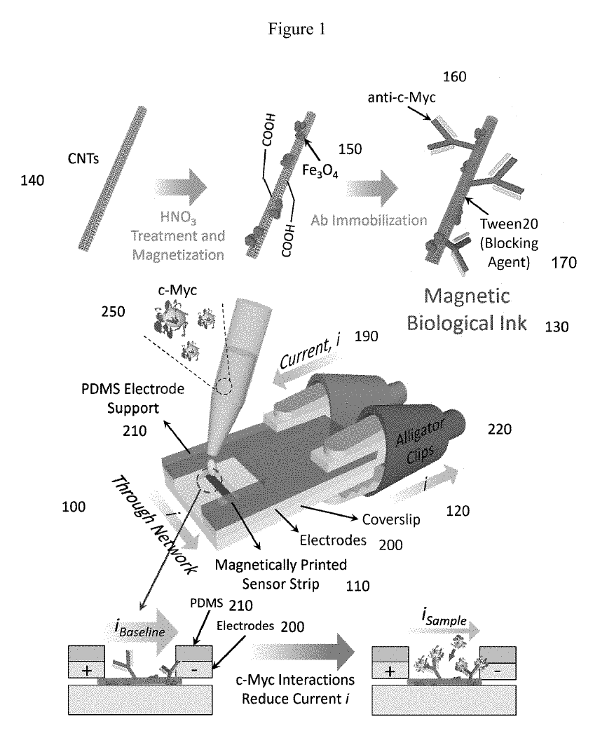

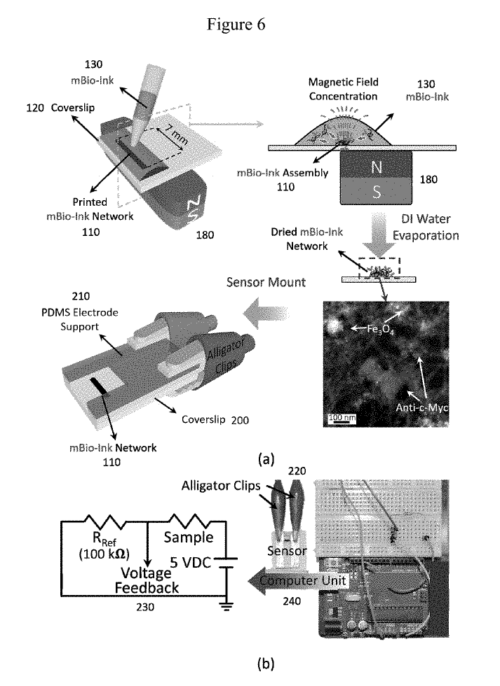

One exemplary embodiment of a biosensor of the application is shown in FIGS. 1 and 6. In this embodiment, biosensor 100 is comprised of a magnetically printed sensor electrode or strip 110, that is deposited or printed on a substrate, such as glass coverslip 120, in the form of a magnetic biological ink (bio-ink) 130. The magnetic bio-ink comprises MWCNT's 140 that have activated by treatment with HNO₃ and reacted with magnetic nanoparticles 150, an antibody such as anti-c-Myc 160 and treated with a blocking agent 170, such as Tween20, and is organized into a strip using external magnetic field 180. After the supernatant of the ink (DI) evaporates, the sensor electode 110 is connected to an external circuit using electrodes 200. In some embodiments, the electrodes are comprised in a polydimethylsiloxane support layer 210 that comprises an insulting gap that is bridged by the sensor electrode 110. In some embodiments the electrodes 200 are connected into the external circuit using a conducting connecting devices such as alligator clips 220 and the like. A voltage divider 230 with a reference resistor R_{ref} of 100 kΩ controlled by a computer 240 monitors current 190 changes that measure the biosensor responses to the various samples 250 that are deposited on it.

Accordingly, the present application includes a biosensor for detecting a target analyte comprising:
a substrate;
a conductive layer on a surface of the substrate;
the conductive layer forming at least one pair of electrodes with an insulating gap between the at least one pair of electrodes;
a sensing electrode bridging the insulating gap, the sensing electrode comprising carbon nanotubes (CNTs) functionalized with magnetic nanoparticles and one or more capture agents;
an external circuit for receiving and/or processing of an electrical signal from the electrodes; and
a detector that detects a change in current through the electrodes resulting from a selective binding interaction between the one or more capture agents and the target analyte.

In some embodiments, the substrate comprises more than one sensor of the application. In some embodiments, the substrate comprises a plurality of biosensors of the application. In some embodiments, the substrate comprises 1 to 100, 1 to 50, 1 to 25, 1 to 10 or 1 to 5 biosensors of the application.

The present application also includes methods of making the biosensors of the application. Accordingly, in some embodiments there is included a method of making a biosensor comprising

(1) preparing a magnetic bio-ink comprising an aqueous solution of mCNTs functionalized with one or more capture agents by:
(a) treating CNT with an oxidizing agent to form reactive functional groups selected from carboxylic acids, aldehydes and alcohols on a surface of the CNTs to provide activated CNTs (aCNTs);
(b) combining the aCNTs with magnetic nanoparticles to provide magnetized CNTs (mCNTs) comprising unreacted carboxylic acids, aldehydes and alcohols;
(c) combining the mCNTs with a capture agent comprising one or more functional groups that form a covalent bond with the unreacted carboxylic acids, aldehydes and alcohols; and
(d) treating the mCNTs from (c) with a blocking agent;
   (2) depositing the magnetic bio-ink onto a substrate;
   (3) forming the magnetic bio-ink into a sensing electrode located in a position electrically connected to an external circuit using an external magnet; and
   (4) allowing the magnetic bio-ink to dry and removing the external magnet.

In some embodiments, the magnetic bio-ink is deposited onto a substrate using any known technique, for example using a pipette, syringe or dropper, or using a printer, such as an inkjet printer or 3D printer.

The present application also includes methods of detecting target analytes using the biosensors of the application. Accordingly, the present application also includes a method of detecting a target analyte comprising:
(a) depositing a sample suspected of comprising the target analyte onto the sensing electrode of a biosensor of the application; and
(b) observing the current through the sensing electrode using the detector,
   wherein a change in current through the sensing electrode in the presence of the sample compared to a control indicates that the sample contains the target analyte.

In some embodiments, the current through the sensing electrode is observed at a time period of about 30 seconds to about 60 seconds after the sample is deposited onto the sensing electrode.

In some embodiments, the control is a blank sample that does not contain the target analyte.

In some embodiments, the biosensor of the application is used in early pathogen detection, in diagnosis of diseases and/or in drug discovery.

### III. Examples

### Materials and Reagents.

Multiwalled CNTs produced by CVD with purity >95%, outside diameters of 20-30 nm, inside diameters of 5-10 nm and lengths between 0.5-2.0 μm were purchased from US Research Nanomaterials. Other reagents used included ferric chloride hexahydrate (FeCl₃.6H₂O, 97-102%, Alfa Aesar), ferrous chloride tetrahydrate (FeCl₂.4H₂O, 98%, Alfa Aesar), nitric acid (HNO₃, 68-70%, CALEDON), and ammonium hydroxide (NH₄OH, 28-30%, CALEDON). C-Myc Ag (Abcam, Cambridge, Mass., USA) had a molecular weight of 49 kDa (49,000 g mol⁻¹). Bovine serum albumin (BSA) (Sigma Aldrich, Oakville, Ontario, Canada) was used as a negative control. All reagents were used as received without further purification. The NdFeB, Grade N52 magnets were purchased from K&J Magnetics Inc (25.4×6×6 mm). The electrode support was fabricated using polydimethylsiloxane (PDMS) and a curing agent (Sylgard 184 kit, Dow Corning). The coverslips (Fisher Scientific, 12-540-B) had dimensions of 22×22×2 mm.

### Characterization Methods and Instruments.

XRD analysis of CNT and MWCNT powder samples was performed using a Bruker D8 Discover instrument comprising a Davinci™ diffractometer operating at 35 kV and 45 mA using Co-Kα radiation (λ_{avg} 1.79026 Å). Bruker's DIFFRAC.Eva V3.1 and TOPAS software were used for the analysis and semi-quantitative estimation of the sample composition. TEM and EELS spectroscopy were conducted with a JEOL 2010F field emission microscope, where the samples were suspended in ethanol, dripped on to a TEM grid and then wicked off with a tissue-wipe. Optical and fluorescence (Enhanced Green Fluorescent Protein, EGFP) microscopy were conducted using a Zeiss Axio Observer.Z1. Magnetization measurements were performed using a SQUID magnetometer at room temperature.

### Production, Purification and Quantification of Anti-c-Myc Monoclonal Abs.

9e10 hybridoma cells were used to produce anti-c-Myc Abs that were then purified with a centrifugal filter to remove fetal bovine serum (FBS) from the cell culture media (Amicon® Ultra-4 Centrifugal Filter with 3k molecular weight limit, and Amicon® Ultra-15 Centrifugal Filter with 100k molecular weight limit). The purified anti-c-Myc Abs were analyzed with SDS-PAGE and their quantification was performed with a Qubit 2.0 Fluorometer. Concentration of the anti-c-Myc Ab suspension resulted in a 0.5 mg/mL concentration.

### Example 1: Two-Step Functionalization of Multiwalled Carbon Nanotubes: Magnetite and Anti-cMyc

The MWCNTs were functionalized in the manner previously reported in Abdalla, A. M.; Ghosh, S.; Puri, I. K., Decorating carbon nanotubes with co-precipitated magnetite nanocrystals. Diamond and Related Materials 2016. Briefly, 1 g of MWCNTs was activated by dispersing it in 200 mL of concentrated nitric acid. The suspension was sonicated for 4 h in a sonication bath (VWR International, Model: 97043-936). The activated MWCNTs (aMWCNTs) were subsequently washed several times with deionized (DI) water, filtered, washed again and finally dried in a vacuum oven. Magnetite nanoparticles (MNPs) were co-precipitated onto the aMWCNTs by stoichiometric calculations to obtain a Fe₃O₄:aCNTs magnetization weight ratios γ=0.1, 0.2, and 0.4 (w/w). For γ=0.4, a 0.92 g FeCl₃.6H₂O and 0.36 g FeCl₂.4H₂O mixture was dissolved in 160 mL of degassed DI water. This was followed by ultrasonic dispersion of 1 g of the aMWCNTs for 10 mins with a probe sonicator (Qsonica, Model: Q500 with ¼″ micro-tip at 35% power) and subsequently for 50 minutes in a sonication bath at 50° C. A 2 ml 30% ammonia solution was slowly introduced as a precipitant to increase the pH to 9. The magnetized MWCNTs thus produced were washed several times until pH 7 was reached, and then filtered and dried in a vacuum oven for 1 hr. In the case of adsorbed MNPs on the surface of MWCNTs, a previous methodology¹² allowed the entanglement of magnetite and MWCNTs, yielding γ=0.4.

### Example 2: Immobilization of Anti-c-Myc Abs on MWCNTs

Following activation and magnetization of the MWCNTs, Ab immobilization was performed. For each mg of MWCNTs contained in the magnetized MWCNTs, 2 mL of deionized water was used as media to disperse the precursor magnetic ink in solution with a probe sonicator (15 seconds, 30% amplitude). An anti-c-Myc to MWCNT weight ratio β=2.5×10⁻⁴ value was selected and an appropriate amount of Ab (0.5 μL, 0.5 mg mL⁻¹) was added to the magnetized MWCNTs (1.4 mg) in solution. The mixture was incubated for 1 hour at room temperature, inverted gently every five minutes to maintain the suspension, or when sedimentation of the magnetic biological ink was observed. Following incubation, the supernatant was removed.

A blocking procedure was performed to prevent non-specific binding of Ag molecules to the ink. Blocking of the MWCNT surface ensures that the detected signal is directly related to the specific Ag-Ab interaction, reducing noise that may originate due to adsorption of non-specific molecules. For every 1 mg of activated MWCNTs, 2 mL of blocking solution (0.1% Tween 20 in deionized water) was added to the magnetic biological ink. The ink was blocked for a half hour at room temperature, inverted gently every five minutes to ensure saturation of the nanoparticle surfaces in the ink. Following incubation with the blocking solution, the ink was washed three times in deionized water (DI). A final concentration of 10 mg/mL was obtained by adjusting the amount of DI water. The same approach was followed for the case when MNPs and Ab were adsorbed on the MWCNT surfaces.

### Example 3: Electrical Circuit, Sensor Assembly and Sensing

To investigate the sensing capability of the dried ink, a voltage divider circuit was used with a reference resistance of R_{ref}=100 kΩ. A square PDMS (2.5×2.5×0.3 cm) section was used to support two aluminum foil electrodes. The PDMS was fabricated using a mold with a mixture of the PDMS precursor and PDMS curing agent (10:1 w/w), which was subsequently degassed for 20 min and cured at 70° C. in an oven. The electrodes were separated by 5 mm and fixed to the PDMS support using double-sided tape. A cutout (1×0.5 cm) through the PDMS support was centered between the electrodes to allow sample deposition on the ink strip. The electrodes were wrapped around this support to provide electrical access with alligator clips. The PDMS electrode assembly was positioned on the top of the sensor, while the alligator clips held the sensor assembly together mechanically. This ensured good connection between the ink network and the aluminum electrodes. Each electrode covered a ˜1 mm section of the sensor, leaving another 5 mm ink strip exposed for sample deposition and therefore Ag detection. The printed sensor of resistance Rₛ was connected in series with R_{ref} A PLC (Arduino Uno) supplied the circuit with a 5 V DC power supply, while an analog feedback voltage allowed the PLC and computer unit to interpret and sample the current i passing through the circuit at a frequency of 10 Hz. After each test, the electrodes were wiped with ethanol (100%) and left to dry to ensure that no cross contamination occurred.

Results and Discussion

The detection of c-Myc Ags was chosen as a proof of concept for the mbio-ink synthesis and application. Recognized by the anti-c-Myc primary Ab, the c-Myc Ag is over-expressed in cancerous tumour cells, where a high expression of c-Myc Ag can accelerate tumour progression, characteristic of malignant phenotypes, potentially earning value as a cancer biomarker to predict tumour behaviour.

The employment of a magnetic field to print the mBio-Ink into electrically conductive networks, facilitating the integration with an external circuit via the use of a simple electrode system, was next demonstrated. The addition of a sample containing c-Myc Ags initiates specific Ag-Ab interactions. When the sensor is connected to an external power source, reduction in current, caused by such interactions, is interpreted by a programmable logic controller (PLC) as a c-Myc detection signal. The rapid detection technique, and generic benchtop fabrication method, allows for scale up, while the small volumes required and magnet independent electrical measurements renders the mbio-ink attractive for drug screening and disease detection applications. The natural ink form of the mbio-ink can equally find applications in inkjet and 3D printed biosensors.

### Functionalization of MWCNTs

MWCNTs were first covalently functionalized by reactive molecular groups when treated with concentrated HNO₃, forming COOH, C═O, and C—OH functional groups that are covalently linked to the MWNT scaffold. The functional groups serve as nucleation sites for the growth of co-precipitating magnetite nanocrystals (Fe₃O₄). The low yield of MNPs lends a chance for active carboxylic groups to remain post magnetization. Thus, subsequent addition of anti-c-Myc is thought to have an increased chance of covalently bonding—through a condensation reaction between the Ab's amine groups and the remaining carboxylic groups—on the surface of the mMWNTs. The MWNT-Fe₃O₄-Ab hybrid nanoparticles are dispersed in an aqueous solution of 0.1% Tween 20 (polysorbate 20). As a blocking agent, Tween 20 coats remaining exposed carbon nanotube surface, and prevents non-specific Ag from interacting with the nanotubes.

### Magnetization of Multi-Walled Carbon Nanotubes

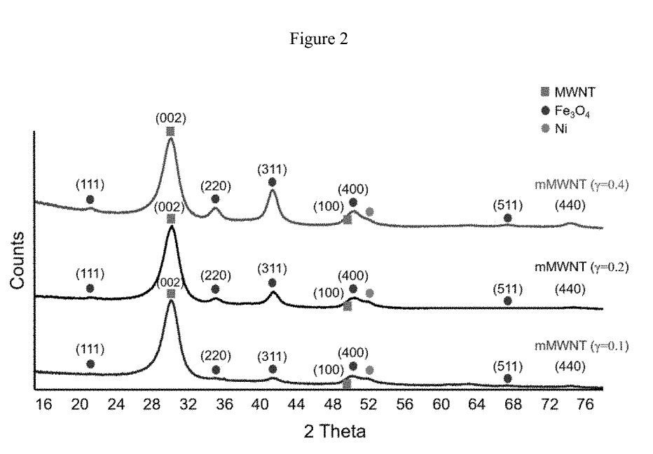

The XRD analysis presented in FIG. 2 corresponds to dried mMWCNTs at three different Fe₃O₄ to MWNTs weight ratio γ=0.1, 0.2, and 0.4, and respectively shows that all samples consist only of the intended magnetite and carbon phases. Use of the powder diffraction file (PDF) database, available through the Eva software, qualitatively confirms that all three samples contain hexagonal carbon (carbon nanotubes, PDF No. 00-058-1638) and the spinel magnetite phase (Fe₃O₄, PDF No. 01-071-6336). The average size of Fe₃O₄ nano-crystals is calculated by applying Scherrer equation at the highest diffraction peak (311).

For all three samples, the MNP size lies in the narrow range from 8.6-10.3 nm. Further, as previously shown using Bragg's Law, [5] the calculated average lattice parameters of the Fe₃O₄ of 8.403, 8.396 and 8.404 Å for γ=0.1, 0.2, and 0.4 respectively agree with the anticipated values for magnetite (8.394 Å, JCPDS No. 79-0417; and 8.400 Å, COD card No. 1011084).

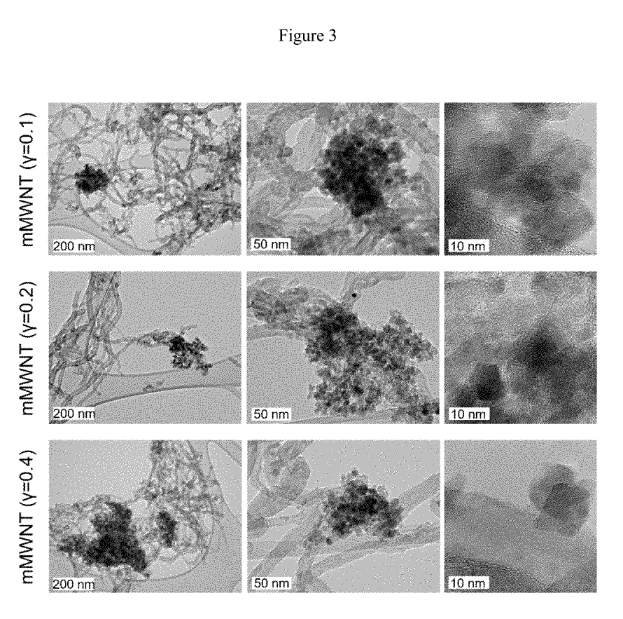

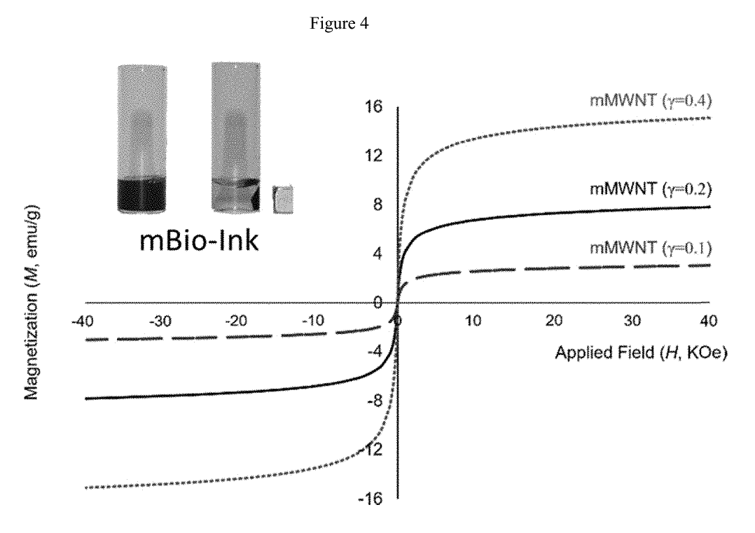

With reference to FIG. 3, TEM images at various magnifications show successful decoration of the MWCNT surfaces by magnetite nanoparticles at all investigated values of γ. Increasing γ from 0.1 to 0.4 raises the decoration density. However, all MNPs have high crystallinity with a narrow crystallite size distribution of ˜10 nm. Because of their small size, magnetite nanoparticles are expected to be superparamagnetic with a high saturation magnetization Mₛ=60-80 emu/g. By integrating them with diamagnetic MWCNTs, the same superparamagnetic behavior is retained in the conjugate material as shown in FIG. 4. All measured hysteresis loops indicate no remanence or coercive field and have magnetic saturations Mₛ=3.03, 7.79, 15.09 emu/g for γ=0.1, 0.2 and 0.4 respectively. Higher Mₛ leads to stronger magnetic response of the conjugate material, which is helpful for magnetic printing and patterning applications. The results in FIG. 4, therefore illustrate that higher magnetic responses of the mMWCNT nanomaterial are directionally proportional to increasing γ (magnetite content) values.

### Antibody Immobilization

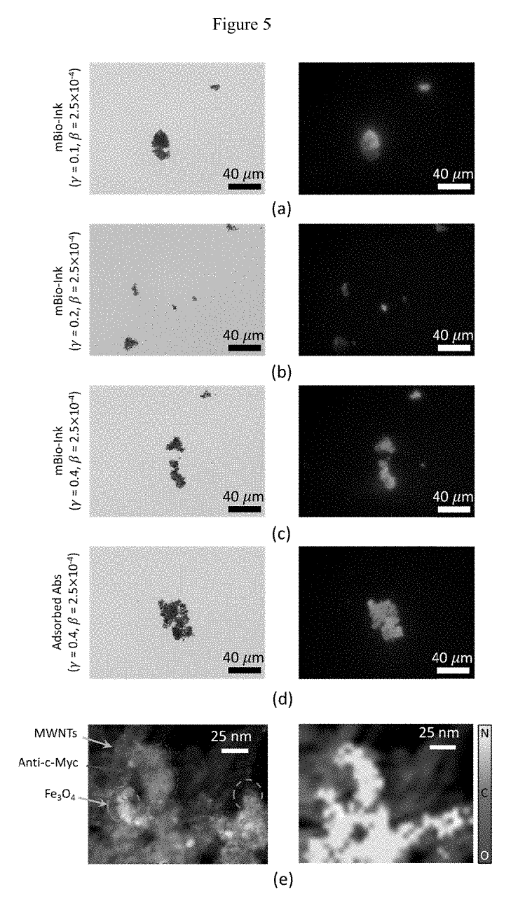

The binding of Ab molecules to the MWCNTs was visualized using fluorescent microscopy. Secondary Ab, fluorescein isothiocyanate (FITC)-conjugated donkey antimouseIgG H&L was used to create a fluorescent ink, for which the Ab:MWCNT weight ratio was β=2.5×10⁻⁴. No inherent fluorescence of MWCNTs or MNPs was observed in samples with γ=0.1, 0.2, and 0.4 and no Ab conjugation, i.e. when the anti-c-Myc to MWCNTs weight ratio β=0 (see FIG. 5(e)). The presence of fluorescence only on mMWCNTs indicates that Abs were successfully immobilized in all cases. Further, the absence of fluorescence in the supernatant for all samples of γ values tested validates the attachment of Abs to the surface of mMWCNTs. The fluorescence intensity is invariant to the Fe₃O₄:MWCNT weight ratio yin the range 0.1-0.4, as shown FIG. 5(a)-(c), i.e., the magnetization of MWCNTs has a negligible impact on Ab immobilization on the nanotube surfaces. Hence, MWCNTs magnetized with γ=0.4, which exhibit robust magnetic response, are used to fabricate the biosensor strip.

The MWCNT surfaces can functionalized with anti-c-Myc Abs through two pathways: (1) covalent attachment and (2) physical adsorption. The relatively low MNP yield from the magnetization of acid-treated MWCNTs allows some active carboxylic groups to remain. Hence, subsequent addition of anti-c-Myc allows the Ab to become covalently bonded to the MWCNTs even without intermediate reagents through a condensation reaction between the Ab amine groups and the remaining carboxylic groups on the mMWCNT surfaces. Alternately, Abs can also be physically adsorbed on non-acid treated MWCNT surface, however Ab-Ag binding cannot be determined using the biosensor since, in this case, the lack of carboxylic acid groups on the surface prevents covalent bonding of Abs, and the Abs, instead, become simply adsorbed to the surface. This latter method relies on the random interactions between the Abs and as-manufactured mMWCNTs, which become physically bonded, possibly through dipole-dipole interactions. Further, this lack of carboxylic acid groups, equally prevents any covalent attachment of the MNPs, which can only be adsorbed, after their separate synthesis, though a simple sonication step with the MWCNTs. The nature of Ab immobilization between the two immobilization methods cannot be visually distinguished in the fluorescence images shown in FIG. 5(a)-(c) and 5(d) depicting the covalently-bonded and adsorbed cases respectively. Qualitatively, the similar fluorescence implies comparable numbers of immobilized Abs in both cases, suggesting negligible differences between covalently bonded and adsorbed cases in selective immobilization of Abs on the surface of mMWCNTs. The results also suggest that while the mbio-ink has an increased chance of covalently bonded Abs, it also includes adsorbed ones.

Anti-c-Myc Ab-conjugated mbio-ink was used for device testing. The purified anti-c-Myc used shows comparable characteristics to commercial anti-c-Myc obtained from 9e10 hybridoma cells, suggesting similar sensor response to commercial anti-c-Myc. Since anti-c-Myc Abs are non-fluorescent optical means cannot be used for visualization. FIG. 5(f) depicts a scanning transmission electron microscopy (STEM) micrograph and its corresponding electron energy loss spectrum (EELS), which highlights the locations where elemental C, O and N are present. O represents the presence of Fe₃O₄ nanoparticles. Since C is present in both carbon nanotubes as well as Abs, N is mapped to highlight the presence of anti-c-Myc Abs. While the result cannot differentiate between adsorbed and covalently bonded Abs, the general structure of the network is seen as a mesh foundation of MWNTs hosting MNPs and Abs, where the MWCNT mesh produces the electrical path and the Abs are Ag receptor sites.

### Sensor Printing

To explore the specificity and bio-sensitivity of the device, a sensor is first printed using 10 μL of anti-c-Myc conjugated mbio-ink deposited on the cover slip, see FIG. 6. A magnet 180 is place underneath the cover slip 120, such that the deposited mbio-ink 130 is attracted to one of the magnet's edges. The edges are used since they provide a magnetic field concentration, creating a dense conductive network. The resultant printed mbio-ink sensor has average dimensions of 7 mm±0.9 mm in length and 1.5 mm±0.2 mm in width (n=25). After a short 20 min drying time at room temperature in the presence of the magnetic field. When the dispersing medium (DI water) had evaporated, dried sensor strips remained on the coverslip, held in place by Van de Waals and electrostatic forces. Despite visual observations of cracks, strips at different times exhibited identical sensing responses to various samples.

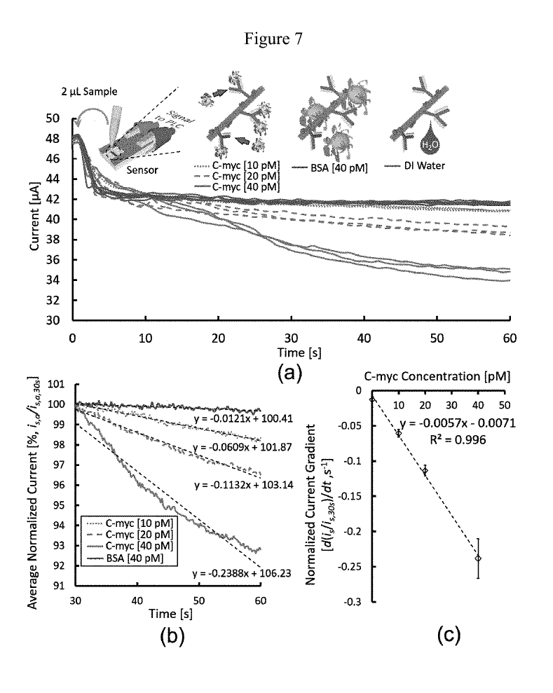

Typically, each sensor consists of ˜100 μg of MWCNTs, ˜40 μg of Fe₃O₄ and ˜25 ng of anti-c-Myc Ab, i.e., the material usage per sensor is small. Hence, the material cost of a printed sensor is lower than 20 cents (Canadian). The sensor is integrated with electrodes using a polydimethylsiloxane (PDMS) support 210 and alligator clips 220 that connect the strip to an electrical circuit. With a reference resistance R_{ref}, an external circuit is used to measure real-time current changes when samples are deposited on the biosensor, see FIG. 7(a). A voltage divider circuit 230 was used to convey information about the change in current i 190 that occurs during sample testing.

### Sensing Measurements

Two types of tests were performed, 1) transient sensor response where a sample is deposited once on the surface of the printed biosensor strip, and 2) sensor response to successive sample addition where equal amounts of the sample are deposited repeatedly after specific intervals on the sensor surface. FIG. 7(b) shows the transient response of the sensors to 2 μL samples of purified c-Myc with concentrations of 40, 20, and 10 pM, as well as DI water and bovine serum albumin (BSA) with a concentration of 40 pM. The BSA was used here as a nonspecific Ag and therefore was a negative control. Each sample was repeated three times, each time with a newly printed sensor. It can be seen from FIG. 7(b) that the baseline current value i_{b}≈47 μA, corresponding to the dry sensor i.e. following evaporation of the mbio-ink solvent (DI water), changes rapidly for all samples at time t=2 s when the sample is introduced to the sensor. For the DI water and BSA samples, the curves are almost superimposed, moreover, a plateau seems to occur in both cases immediately after sample addition. However, for the c-Myc cases, a gradual decrease in the sample current iₛ continues to occur to values below that of the DI water and BSA curves. Therefore the biosensor responds differently to c-Myc sample deposition due to the specific interaction of anti-c-Myc Ab with the c-Myc Ag. This increases the electrical resistance of the sensor, which in turn decreases iₛ. Nonspecific interactions of anti-c-Myc with DI water and BSA do not decrease iₛ as significantly below its initial value. The relatively slow Ag-Ab binding induce the gradual current reduction.

This response highlights the Ag-Ab binding kinetics that occurs in the mbio-ink network. The significant difference in response between c-Myc positive and negative control samples can be attributed to the increased chance of covalently bonded Abs in the mbio-ink. When power is supplied to the sensor network, interactions with anti-c-Myc, such as in the presence of specific c-Myc Ags, increase the resistance locally and can cause current reductions. Further, because a certain time period passed for the majority of c-Myc to bind, the reduction in current is gradual and will eventually reach a plateau. Comparatively, when nonspecific interactions with the anti-c-Myc occur, the departure from i_{b} is not as severe and plateaus rapidly, as demonstrated with the DI water and BSA samples. Thus the sensor response in FIG. 7(b) highlights the specificity of the mbio-ink in detecting c-Myc.

Since this Ag-Ab binding action causes a reduction in current through the network, higher Ag concentrations would result in more binding per unit time and thus causes a larger reduction in current, leading to sharper current gradients diₛ/dt. To demonstrate this, consider the values of iₛ in FIG. 7(b) at t=60 s, for c-Myc samples with concentrations 40, 20, and 10 pM, which are 34.6±0.5 μA, 38.8±0.4 μA, and 40.9±0.1 μA respectively. This confirms that current reduction is proportional to c-Myc concentration. In addition, at time 30 s<t<60 s, a relatively stable gradient diₛ/dt can be seen for all curves, which visually varies between each case. To better interpret the current gradients, FIG. 7(c) shows the normalized averaged current i_{savg}/i_{savg,30s} where i_{savg} is the average current of all three repetitions of the sample, and i_{savg,30s}=i_{savg} at time t=30 s. A linear regression equation is then fitted to each of the curves and the line equations are plotted (DI water not shown). FIG. 7(c) shows that while the BSA curve remains relatively constant, the magnitude of the gradients for the c-Myc samples are directionally proportional to the c-Myc concentration. FIG. 7(d) illustrates a linear correlation between the normalized current gradients and the c-Myc concentrations. Both FIG. 7(c) and FIG. 7(d) are the result of 12 identically printed devices, i.e. 3 for each case of 10 pM, 20 pM, and 40 pM of c-Myc and 40 pM of BSA. By simply monitoring the sensor's transient electrical response, it is possible to rapidly identify c-Myc positive samples with different concentrations. This makes Ag monitoring feasible without using additional reagents and sophisticated electrical equipment that is typical of other biosensors.

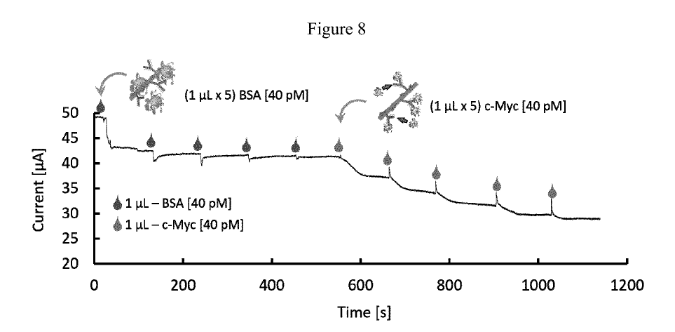

FIG. 8 displays the biosensor response to successive drops of BSA, directly followed by c-Myc. Here, the biosensor demonstrates its sensitivity to its target antigen, even after saturation with using a non-target molecule.

Defect sites present on the surface of MWCNTs restrict electron transport and act as resistance hotspots. Interactions in the vicinity of the defect sites, impost further resistance, which can be conveyed through a reduction in current reported by the external circuitry. After the acidic treatment of MWCNTs, the defect sites become populated with active carboxylic acid groups. As such, these sites are able to host Abs, which can covalently bond to them through a condensation reaction with the Abs amine groups. When the target Ags bind to the Abs, this inflicts more resistance to the electron transport, since the Abs communicate such binding through interactions with the defect sites. It is thought that covalent immobilization of Abs further amplifies such interactions compared to simply adsorbed ones, thereby offering a more robust reporting of target Ags given the biosensor setup. Therefore, in other words, the case of Ags binding to Abs can be considered as indirect interactions with the defect sties, finally increasing the biosensor material's overall resistance.

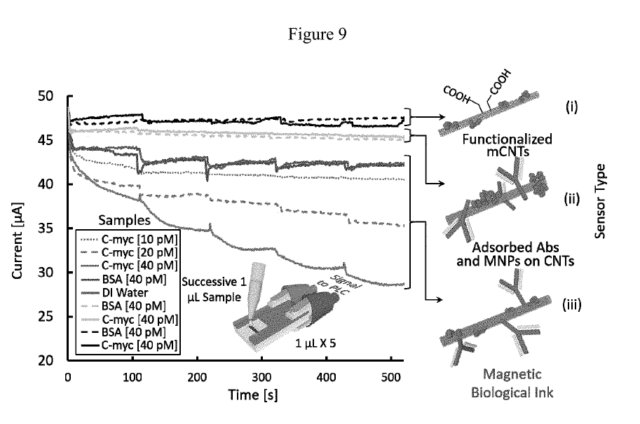

The response of the biosensor to successive 1 μL sample depositions is presented in FIG. 9 for three types of biosensor strips that are printed with three different inks. These inks contain (i) covalently magnetized MWCNTs that have not yet been functionalized with Abs (ink 1), or (ii) MWCNTs that have both Fe₃O₄ nanoparticles and anti-c-Myc Abs adsorbed on their surfaces (ink 2), and (iii) MWCNTs that are covalently magnetized with Fe₃O₄ nanoparticles and contain both covalently bonded and adsorbed anti-c-Myc Abs on their surfaces (ink 3). The functionalized MWCNTs for all three inks are dispersed in DI water containing 0.1% Tween 20.

Biosensors printed with inks 1 and 2 do not discriminate between 40 pM c-Myc Ag and 40 pM BSA, i.e., they produce similar responses for these two samples and it is not possible to positively detect c-Myc by printing biosensor strips with these two inks. The biosensor remains specific to the target antigen in the presence of another protein molecule. When the mbio-ink (ink 3) was used to print the sensor, the differences between specific (c-Myc) samples and non-specific (BSA) samples are clearly apparent, and thus greatly amplified by the mbio-ink. The c-Myc [10 pM] sample shows the least reduction in current, followed by c-Myc [20 pM] and c-Myc [40 pM], compared with the little-changing BSA and DI water responses. The similarity of the BSA to DI water response, confirms the sensor's consistent specificity to c-Myc after a total sample volume of 5 μL was added. Successive additions of c-Myc sample further decreases the current with the first addition causing the most reduction in all c-Myc samples.

To further illustrate the signal amplification role of the mbio-ink, consider the BSA samples in FIG. 9. The baseline current for all samples, i_{b}≈47 μA, quickly reduces to different average values for different sensor types. When the sensors are left to dry after printing, anti-c-Myc Abs equally dry up, resulting in a conformational change of the protein structure. When a sample is added to the sensor, the DI water present in all samples allows the anti-c-Myc to return to its native structure. This reversion can trigger a current reduction by locally increasing resistance through physical interaction with the mMWCNT network. This potentially correlates to the initial current reductions observed in all tests. In the case of mMWCNTs this reduction is lowest, since there are no anti-c-Myc present, and is attributed to nonspecific interactions and network response to DI water. When the anti-c-Myc Abs are present, as in the adsorbed case, such reduction is larger than the mMWCNTs case. However, the response is lower than that of the mbio-ink. The mbio-ink fabrication method allows for the anti-c-Myc to amplify nonspecific interaction such as with BSA. However, the amplification is significantly greater for specific Ag-Ab binding interactions, as observed in the c-Myc samples in FIG. 9. This lends enough contrast between specific and nonspecific interactions to confidently distinguish and identify the target Ag. This can be further emphasized from FIG. 9 when acknowledging the signal difference between c-Myc [40 pM] and BSA [40 pM] at t=500 s being 12.88 μA for the mbio-ink sensor compared to the negligible differences of the other sensor types.

The previous non-limiting examples are illustrative of the present application. While the present application has been described with reference the prior examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Abdel Fattah, A. R.; Majdi, T.; Abdalla, A. M.; Ghosh, S.; Puri, I. K., Nickel Nanoparticles Entangled in Carbon Nanotubes: Novel Ink for Nanotube Printing. ACS Appl. Mater. & Interfaces 2016, 8 (3), 1589-93.
2. Masotti, A.; Caporali, A., Preparation of magnetic carbon nanotubes (Mag-CNTs) for biomedical and biotechnological applications. Int J Mol Sci 2013, 14 (12), 24619-42.
3. Meng, L.; Fu, C.; Lu, Q., Advanced technology for functionalization of carbon nanotubes. Progress in Natural Science 2009, 19 (7), 801-810.
4. Balasubramanian, K.; Burghard, M., Chemically functionalized carbon nanotubes. Small 2005, 1 (2), 180-92.
5. Korneva, G.; Ye, H.; Gogotsi, Y.; Halverson, D.; Friedman, G.; Bradley, J. C.; Kornev, K. G., Carbon nanotubes loaded with magnetic particles. Nano Lett 2005, 5 (5), 879-84.
6. He, H.; Gao, C., Synthesis of Fe3O4/Pt Nanoparticles Decorated Carbon Nanotubes and Their Use as Magnetically Recyclable Catalysts. Journal of Nanomaterials 2011, 2011, 1-10.
7. Abdel Fattah, A. R.; Ghosh, S.; Puri, I. K., Printing Microstructures in a Polymer Matrix using a Ferrofluid Droplet J. Mag. Mag. Mater. 2016, 401, 1054-1059.
8. Ganguly, R.; Puri, I. K., Field-assisted self-assembly of superparamagnetic nanoparticles for biomedical, MEMS and BioMEMS applications. Adv. in Appl. Mech. 2007, 41, 293-335.
9. Ganguly, R.; Puri, I. K., Microfluidic transport in magnetic MEMS and bioMEMS. Wiley Interdisciplinary Rev.: Nanomed. and Nanobiotech. 2010, 2, 382-399.
10. Puri, I. K.; Ganguly, R., Particle Transport in Therapeutic Magnetic Fields. Annual Review of Fluid Mechanics 2014, 46 (1), 407-440.
11. Abdel Fattah, A. R.; Ghosh, S.; Puri, I. K., Printing three-dimensional heterogeneities in the elastic modulus of an elastomeric matrix. ACS Appl Mater Interfaces 2016.
12. Tsai, P. J.; Ghosh, S.; Wu, P.; Puri, I. K., Tailoring Material Stiffness by Filler Particle Organization. ACS Applied Materials & Interfaces 2016, 8 (41), 27449-27453.
13. Qu, S.; Wang, J.; Kong, J.; Yang, P.; Chen, G., Magnetic loading of carbon nanotube/nano-Fe(3)O(4) composite for electrochemical sensing. Talanta 2007, 71 (3), 1096-102.
14. Liu, Z.; Wang, J.; Xie, D.; Chen, G., Polyaniline-coated Fe3O4 nanoparticle-carbon-nanotube composite and its application in electrochemical biosensing. Small 2008, 4 (4), 462-6.
15. Pérez-López, B.; Merkoci, A., Magnetic Nanoparticles Modified with Carbon Nanotubes for Electrocatalytic Magnetoswitchable Biosensing Applications. Advanced Functional Materials 2011, 21 (2), 255-260.
16. Zarei, H.; Ghourchian, H.; Eskandari, K.; Zeinali, M., Magnetic nanocomposite of anti-human IgG/COOH-multiwalled carbon nanotubes/Fe(3)O(4) as a platform for electrochemical immunoassay. Anal Biochem 2012, 421 (2), 446-53.

1. A biosensor for detecting a target analyte comprising:
an external circuit;
a sensing electrode that is electrically connected to the external circuit;
the sensing electrode comprising carbon nanotubes (CNTs) functionalized with magnetic nanoparticles (MNPs) and one or more capture agents; and
a detector that detects a change in current through the sensing electrode resulting from a selective binding interaction between the one or more capture agents and the target analyte.

2. The biosensor of claim 1, further comprising a transmitter for sending data obtained by the biosensor to a remote sensor and a power source.

3. The biosensor of claim 1, wherein the external circuit is a voltage divider circuit.

4. The biosensor of claim 1, wherein the CNTs have been activated to provide functional groups on at least part of the surface of the CNTs for attachment of the magnetic nanoparticles and capture agents.

5. The biosensor of claim 1, wherein the magnetic nanoparticles are magnetite nanocrystals (Fe₃O₄).

6. The biosensor of claim 5, wherein the Fe₃O₄:CNT weight ratio (γ) is from about 0.05 to about 1.

7. The biosensor of claim 1, wherein the capture agent:CNT weight ratio (β) is about 2.5×10⁻² to about 2.5×10⁻⁴.

8. The biosensor of claim 1, wherein the target analyte is selected from a biomolecule and any material comprising a biomolecule.

9. The biosensor of claim 1, wherein the capture agent is any molecule that contains a functional group that will covalently bond to the activated CNTs and that will specifically interact with the target analyte so that the target analyte becomes immobilized on the sensing electrode.

10. The biosensor of claim 1, wherein the target analyte is an antibody and the capture agent is the antigen that specifically binds to that antibody.

11. The biosensor of claim 1, wherein the target analyte is an antigen and the capture agent is the antibody that specifically binds to that antigen.

12. The biosensor of claim 1, wherein the CNTs functionalized with magnetic nanoparticles (MNPs) and one or more capture agents are treated with a blocking agent.

13. The biosensor of claim 1, wherein the sensing electrode is located so that it bridges a gap between the two further electrodes to complete the external circuit.

14. The biosensor of claim 13, further comprising a support and the sensing electrodes, and any further electrodes are on a surface of the support.

15. The biosensor of claim 1, wherein a magnet is not used in the biosensor during detection of the target analyte.

16. The biosensor of claim 1, wherein the change in current through the sensing electrode resulting from a selective binding interaction between the one or more capture agents and the target analyte is a decrease in current that is proportional to the concentration of the target analyte in the sample.

17. The biosensor of claim 1, comprising:
a substrate;
a conductive layer on a surface of the substrate;
the conductive layer forming at least one pair of electrodes with an insulating gap between the at least one pair of electrodes;
a sensing electrode bridging the insulating gap, the sensing electrode comprising CNTs functionalized with magnetic nanoparticles and one or more capture agents;
an external circuit for receiving and/or processing of an electrical signal from the electrodes; and
a detector that detects a change in current through the electrodes resulting from a selective binding interaction between the one or more capture agents and the target analyte.

18. The biosensor of claim 1, wherein the CNTs are multiwall carbon nanotubes (MWCNTs).

19. A method of making the biosensor comprising:
(1) preparing a magnetic bio-ink comprising an aqueous solution of mCNTs functionalized with one or more capture agents by:
(a) treating CNT with an oxidizing agent to form reactive functional groups selected from carboxylic acids, aldehydes and alcohols on a surface of the CNTs to provide activated CNTs (aCNTs);
(b) combining the aCNTs with magnetic nanoparticles to provide magnetized MWCNTs (mCNTs) comprising unreacted carboxylic acids, aldehydes and alcohols;
(c) combining the mCNTs with a capture agent comprising one or more functional groups that form a covalent bond with the unreacted carboxylic acids, aldehydes and alcohols; and
(d) treating the mCNTs from (c) with a blocking agent;
(2) depositing the magnetic bio-ink onto a substrate;
(3) forming the magnetic bio-ink into a sensing electrode located in a position on the substrate electrically connected to an external circuit using an external magnet; and
(4) allowing the magnetic bio-ink to dry and removing the external magnet.

20. A method of detecting a target analyte comprising:
(a) depositing a sample suspected of comprising the target analyte onto the sensing electrode of a biosensor of claim 1; and
(b) observing the current through the sensing electrode using the detector,
wherein a change in current through the sensing electrode in the presence of the sample compared to a control indicates that the sample contains the target analyte.
